(19) United States
(12) Patent Application Publication
(10) Pub. No.: US 2009/0018404 A1
(43) Pub. Date: Jan. 15, 2009

(54) Cardiovascular Autonomic Neuropathy Testing Utilizing an Implantable Medical Device

(75) Inventors: Lahn M. Fendelander, Arlington, MA (US); Lizbeth M. Mino, Northfield, MN (US); Aaron R. McCabe, Minneapolis, MN (US)

Correspondence Address: PAULY, DEVRIES SMITH & DEFFNER, L.L.C., PLAZA VII- SUITE 3000, 45 SOUTH SEVENTH STREET, MINNEAPOLIS, MN, 55402-1630, US

(73) Assignee: CARDIAC PACEMAKERS, INC., St. Paul, MN (US)

(21) Appl. No.: 11/776,744

(22) Filed: Jul. 12, 2007

(51) Int. Cl. A61B 5/00

(52) U.S. Cl. 600/301; 600/486; 600/529; 600/547; 600/595

(57) ABSTRACT

A test system and method for cardiovascular autonomic neuropathy that incorporates an implanted medical device. One aspect of the invention relates to a system for performing cardiovascular autonomic neuropathy (CAN) testing in a diabetic patient having an implantable medical device (IMD) that includes a plurality of implantable physiological sensors and that is configured to transmit a wireless signal corresponding to a sensed physiological activity and to receive wireless signals. The system further includes one or more non-implantable physiological sensors, where the non-implantable physiological sensors are each configured to transmit a signal corresponding to a sensed physiological parameter, and a monitor device having a patient interface. The monitor device is configured to interface with a patient, including directing the patient to answer health related questions and use one or more of the non-implantable physiological sensors. The monitor device is also configured to receive signals, including signals from the IMD and the non-implantable physiological sensors. The system is configured to provide an indication of the presence or progression of CAN.

### FIELD OF THE INVENTION

The invention relates to the administration of tests for cardiovascular autonomic neuropathy, and more specifically, to the administration of cardiovascular autonomic neuropathy tests in a patient having an implanted medical device.

### BACKGROUND OF THE INVENTION

Diabetes is a relatively common affliction in which a person's body does not produce or properly use the hormone called insulin that is needed to convert sugar, starches and other food into energy. By some estimates, about seven percent of the population of the United States has diabetes. The symptoms of diabetes are often not recognized, and it is estimated that about one-third of people with the disease do not realize they have it.

Compared to the general population, diabetes is estimated to be even more common within the population of patients who have an implanted cardiac rhythm management device. In one estimate, approximately 11-13% of patients with implantable pacemakers have diabetes, approximately 30-38% of patients having implantable cardioverter defibrillators have diabetes, and approximately 39-45% of patients having cardiac resynchronization therapy (CRT) devices have diabetes.

A common complication of diabetes is called Diabetic Autonomic Neuropathy (DAN). DAN is a condition that develops at some point after onset of diabetes and then progresses slowly over the course of the diabetic disease. By some estimates, almost 100% of patients with diabetes will eventually develop some form of DAN. DAN generally impairs a patient's ability to conduct activities of daily living and lowers quality of life. Moreover, DAN creates a risk of serious complications for the patient, the most important being that it diminishes the patient's ability to sense a hypoglycemic state and also diminishes the patient's ability to sense an incipient heart attack and seek appropriate medical attention. These and other risks create an elevated mortality rate, such that by some estimates the 5-year mortality rate in patients with DAN is three times higher than in diabetic patients without autonomic involvement. Furthermore, DAN also accounts for a large portion of the cost of care of a diabetic patient.

Although the mechanisms that cause DAN are not entirely understood, it is believed that the condition results, at least in part, from diabetic microvascular injury to the small blood vessels that supply nerves. DAN involves injury to a number of different autonomic functions, but one of the most clinically important is cardiovascular autonomic neuropathy (CAN). CAN results in impairment to autonomic control of cardiovascular function, typically resulting in abnormal heart rate and blood pressure reflexes, and is linked to increased patient mortality.

CAN is a degenerative disease that progresses slowly, but inexorably, in a patient. CAN is capable, however, of being managed through intensive glycemic control that can slow its progression. Furthermore, there are a variety of pharmacologic and nonpharmacologic therapies that are available to treat the symptoms of autonomic neuropathy. Therefore, it is desirable to monitor a patient for indications of the presence and degree of CAN in order to be able to properly manage the disease.

Improved techniques for monitoring diabetic patients for CAN are needed. In particular, techniques are needed for monitoring diabetic patients for CAN that are convenient for the patient, are less imposing on the resources of the medical care system, and are readily conducted at regular intervals.

### SUMMARY OF THE INVENTION

A test for cardiovascular autonomic neuropathy is disclosed that incorporates an implanted medical device. One aspect of the invention relates to a system for performing cardiovascular autonomic neuropathy (CAN) testing in a diabetic patient. The system has an implantable medical device (IMD) that includes a plurality of implantable physiological sensors and that is configured to transmit a wireless signal corresponding to a sensed physiological activity and to receive wireless signals. The system further includes one or more non-implantable physiological sensors, where the non-implantable physiological sensors are each configured to transmit a signal corresponding to a sensed physiological parameter, and a monitor device having a patient interface. The monitor device is configured to interface with a patient, including directing the patient to answer health related questions and use one or more of the non-implantable physiological sensors. The monitor device is also configured to receive signals, including signals from the IMD and the non-implantable physiological sensors. The system is configured to provide an indication of the presence or progression of CAN.

Another aspect of the invention relates to a method of testing for cardiovascular autonomic neuropathy (CAN) in a diabetic patient. The method includes providing an implanted medical device (IMD) that includes a plurality of implantable physiological sensors, where the IMD is configured to transmit a wireless signal corresponding to a sensed physiological activity and to receive wireless signals. The method further includes providing a plurality of non-implantable physiological sensors, where the non-implantable physiological sensors are each configured to transmit a signal corresponding to a sensed physiological parameter, and providing a monitor device having a patient interface. The method also includes directing a patient through the patient interface to answer health related questions, interface with one or more of the non-implantable physiological sensors, and to perform a diagnostic procedure. The method further involves receiving signals at the monitor device, including signals from the IMD and the non-implantable physiological sensors, and using an implantable physiological sensor to verify that the patient properly performed the diagnostic procedure.

Another embodiment of the invention is a system for performing testing for cardiovascular autonomic neuropathy (CAN) in a diabetic patient, the system including an implanted medical device (IMD) system that includes a plurality of implantable physiological sensors, the IMD system being configured to transmit a wireless signal corresponding to a sensed physiological activity and receive wireless signals. The system further includes a plurality of non-implantable physiological sensors, where the non-implantable physiological sensors are each configured to transmit a signal corresponding to a sensed physiological parameter. The system further includes a monitor device configured to receive signals including signals from the IMD system and the non-implantable physiological sensors. The monitor device includes a patient interface and is configured to direct a patient through the patient interface to answer health related questions, interface with one or more of the non-implantable physiological sensors, and perform a diagnostic procedure. The IMD system further includes an implantable physiological sensor to verify that the patient properly performed the diagnostic procedure.

The invention may be more completely understood by considering the detailed description of various embodiments of the invention that follows in connection with the accompanying drawings.

### BRIEF DESCRIPTION OF THE DRAWINGS

FIG. 1 is a schematic representation of a medical system including an implantable medical device for testing a diabetic patient for cardiovascular autonomic neuropathy.

FIG. 2 is a schematic representation of functional elements of an embodiment of an implantable medical device.

FIG. 3 is a schematic representation of a CAN testing protocol that utilizes an implantable medical device.

FIG. 4 is a perspective view of an embodiment of a monitor device for use with the present invention.

FIG. 5 is a schematic representation of functional elements of an embodiment of a monitor device.

While the invention may be modified in many ways, specifics have been shown by way of example in the drawings and will be described in detail. It should be understood, however, that the intention is not to limit the invention to the particular embodiments described. On the contrary, the intention is to cover all modifications, equivalents, and alternatives following within the scope and spirit of the invention as defined by the claims.

### DETAILED DESCRIPTION OF THE INVENTION

Cardiovascular autonomic neuropathy (CAN) is one aspect of diabetic autonomic neuropathy (DAN). CAN is a serious complication of diabetes that can create quality of life problems and increases the risk of patient mortality. Because CAN can be managed through appropriate medical treatment, it is important to test diabetic patients for the presence of the condition. Testing is preferably conducted early after a patient is diagnosed with diabetes and then at regular intervals until CAN is identified. Once the existence of CAN is identified, it is desirable to continue to monitor the patient for worsening progression of CAN. However, one problem is that many diabetic patients are not regularly followed by a specialist who is monitoring the patient for CAN, and therefore not all patients receive the appropriate testing at the appropriate intervals. It is desirable to have a CAN testing regime that does not require the oversight or direct involvement of a specialist.

Testing is a key aspect of overall diabetes and DAN management. In one standard treatment protocol, a patient who is diagnosed with Type 1 diabetes is tested for symptoms of CAN five years after diagnosis, although it is possible, and even desirable, to test such a patient earlier and more frequently. It is also standard treatment protocol to test a patient who is diagnosed with Type 2 diabetes for symptoms of CAN immediately after diagnosis. If a patient is tested and the test is negative for symptoms of CAN, then the patient is retested annually until symptoms arise. Once symptoms have been identified, then therapy is initiated, including treatment of symptoms and appropriate further diagnostic tests. A key aspect of control of CAN and DAN is intensive glycemic control. In fact, some studies have shown that intensive glycemic control can reduce the prevalence of the symptoms and effects of DAN by over 50%.

CAN involves degeneration of the autonomic control of cardiac function. Symptoms of CAN include, for example, lack of heart rate variability in response to breathing or exercise, and also the possibility of associated poor exercise tolerance. Furthermore, an excessively rapid resting heart rate (tachycardia) is an indication of the presence of CAN. Some patients having CAN will experience blunted symptoms of coronary artery disease, such as painless ischemia and silent myocardial infarction (MI). These are particularly important aspects of the disease because of the potential that the patient will not realize the presence of a condition that requires immediate medical attention.

A testing protocol for CAN generally includes tests of autonomic control of heart function. CAN tests measure heart rate variability and blood pressure response to various activities and circumstances. The following tests can be used to evaluate the existence and nature of CAN in a diabetic patient. These tests will also be described later herein in the context of administering these tests using certain embodiments of the invention:

(1) Test resting heart rate. A heart rate of greater than 100 beats per minute is abnormal.

(2) Measure beat-to-beat heart rate variation while lying supine and breathing six times per minute. A difference in heart rate of less than 10 beats per minute is abnormal, compared to prior to the patient regulating breathing.

(3) Measure beat-to-beat heart rate variation for inspiration compared to exhalation. An exhalation:inspiration R-R ratio of greater than 1.17 is abnormal. The R-R interval is the amount of time between two consecutive R waves of an electrocardiogram.

(4) Determine heart rate response to standing. Generally, this involves measuring the R-R interval on an electrocardiogram at the fifteenth and the thirtieth beats after standing. An increase in the heart rate of less than 3 percent from the fifteenth to the thirtieth beats is abnormal.

(5) Test heart rate response to the Valsalva maneuver, where the patient forcibly exhales into the mouthpiece of a manometer at a pressure of 40 mm Hg for 15 seconds. A ratio of the longest R-R interval to the shortest of less than 1.2 is abnormal.

(6) Measure systolic blood pressure response to standing, where a first blood pressure measurement is taken when the patient is lying down and a second blood pressure measurement is taken two minutes after the patient stands. A decrease in systolic blood pressure in response to standing of more than 30 mm Hg is abnormal, and a decrease of 10 to 29 mm Hg is borderline.

(7) Measure diastolic blood pressure in response to isometric exercise. This test involves having a patient squeeze a handgrip dynamometer to establish a maximum exertion, and then having the patient squeeze the grip at 30 percent of maximum for five minutes. A rise in diastolic blood pressure of less than 16 mm Hg in the contralateral arm is abnormal.

(8) Conduct electrocardiography and determine a corrected QT interval (QTc). Because the actual QT interval is affected by the patient's heart rate, a corrected QT interval is one that is corrected for the heart rate so that various QT interval measurements can be compared against each other. A QTc of more than 440 ms is abnormal.

(9) Conduct electrocardiography and determine heart-rate variability. Testing for reduced heart-rate variability using electrocardiography over a longer period of time, such as over 24 hours, can reveal CAN earlier than reflex tests, like those determining the body's response to standing. By recording of the heart-rate over a 24-hour period, abnormal circadian rhythms can be detected, thereby revealing problems with sympathovagal activity. One measure of heart rate variability is a frequency domain measure. A common frequency domain method is to apply a discrete Fourier transform to the patient's beat-to-beat interval time series. The result expresses the amount of variation for different frequencies. Several frequency bands of interest have been defined in humans, including the High Frequency band (HF) between 0.15 and 0.4 Hertz. The High Frequency band is driven by respiration and appears to derive mainly from vagal activity. The Low Frequency band is between 0.04 and 0.15 Hz and derives from both vagal and sympathetic activity. The Very Low Frequency band is between 0.0033 and 0.04 Hz. A depressed very-low frequency peak indicates sympatho-vagal imbalance, and a depressed high frequency peak indicates parasympathetic dysfunction. A lowered low-frequency to high-frequency ratio indicates sympathetic imbalance. The exact values expected for the low and high frequency peaks will vary depending on the patient's age and other factors. A description of methods of analyzing heart rate variability for assessing autonomic balance is described in commonly-owned U.S. Pat. Nos. 7,069,070, 7,215,992 and U.S. Published Patent Application 2004-0158295, which patent documents are hereby incorporated herein by reference.

These tests are traditionally performed in a physician's office or other medical facility. Because many of these tests require the patient to perform a certain motion, such as standing up, they typically require the direction and monitoring of a medical professional. Because of the inconvenience, cost, and time involved in conducting these tests, they are often conducted at intervals of about a year or greater. However, it is desirable to reduce the inconvenience, cost, and time involved in conducting the screening tests. This is advantageous to the patient and the medical system. Furthermore, it may as a practical matter allow the screening test to be conducted more often, and in doing so, to identify symptoms of CAN earlier and to provide for earlier and more effective treatment. This earlier and more effective treatment may slow the progression of the disease, thereby reducing mortality and increasing quality of life. The screening tests may also be more accurate.

As discussed above, there tends to be overlap between the population of diabetics and the population of heart patients who have implantable medical devices such as cardiac rhythm management (CRM) devices. The inventors have devised a CAN test protocol that can be executed by or in conjunction with a patient's implantable medical device. This arrangement utilizes some of the capabilities inherent in an implantable medical device, and further incorporates additional sensors and telemetric communications to accomplish a CAN test. The CAN test protocol has the advantage that it can be conducted in a patient's home or other convenient location, and does not require a visit to a medical facility and the direct attention of trained medical personnel. These advantages may, as a practical effect, allow the test to be performed more frequently, thereby leading to earlier diagnosis and treatment. Furthermore, there is less inconvenience to the patient and less expense for the medical system.

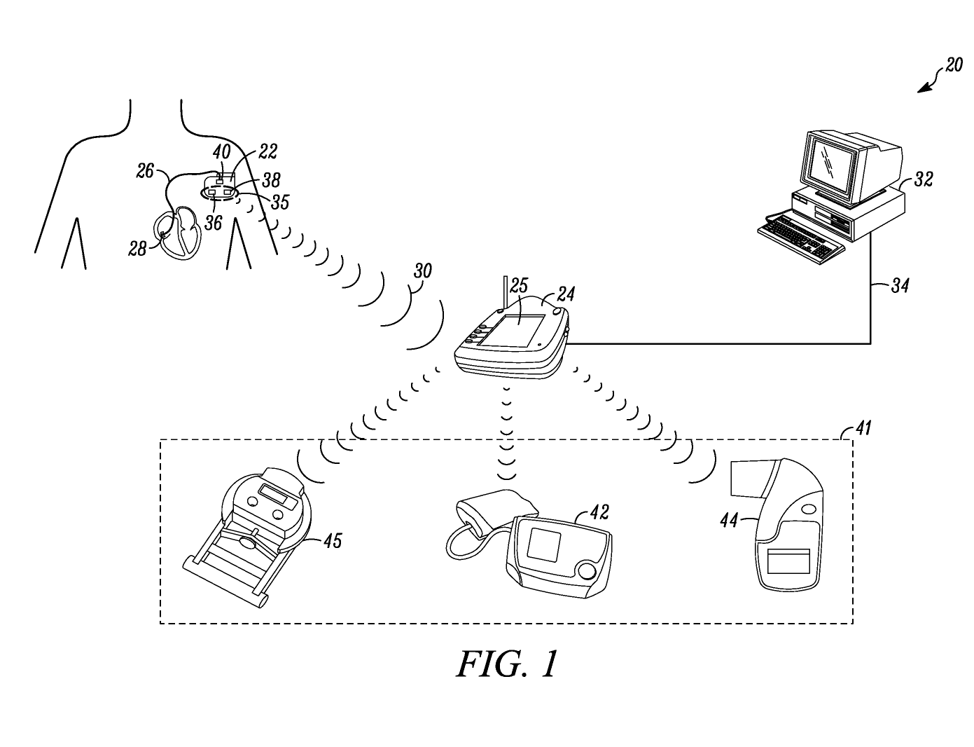

An embodiment of a medical system for testing diabetic patients for CAN that is constructed according to the principles of the present invention is depicted in FIG. 1. The medical testing system 20 includes an implantable medical device 22 such as a cardiac rhythm management (CRM) device 22. CRM device 22 may be, for example, a pacemaker, an implantable cardioverter defibrillator, or a cardiac resynchronization therapy (CRT) device. Other types of CRM devices are usable. CRM device 22 has at least one lead 26 that forms an electrically conductive path to an electrode 28. Electrode 28 is in contact with cardiac tissue and is capable of sensing cardiac electrical activity. A cardiac electrical signal is transmitted from electrode 28 through lead 26 to CRM device 22, where it is received and forms the basis of an electrocardiogram that is indicative of the operation of the patient's heart. Some embodiments of a CRM device have two leads and two electrodes, and other embodiments may have three leads. In some embodiments, a CRM device 22 is further configured to communicate by telemetry with appropriately configured devices outside of the patient's body.

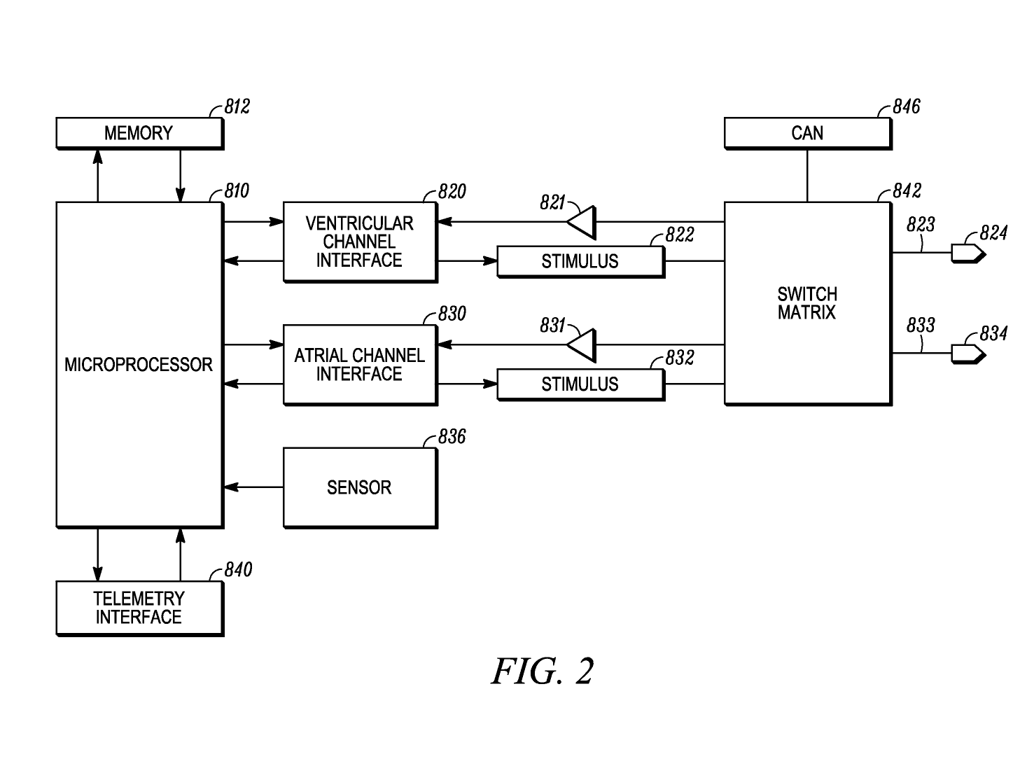

FIG. 2 schematically depicts certain functional elements of an example embodiment of a CRM device 22. A controller senses cardiac events through a sensing channel and outputs pacing pulses to the heart via a pacing channel in accordance with a programmed pacing mode. A microprocessor 810 serves as the controller in this embodiment and communicates with a memory 812 via a bidirectional data bus. The memory 812 typically comprises a ROM or RAM for program storage and a RAM for data storage. The implantable medical device is shown in FIG. 2 as having two leads, each having a tip electrode. Alternatively, bipolar leads are provided with ring and tip electrodes. Other embodiments have one or three leads. In one embodiment, electrode 834 is analogous to electrode 28 depicted in FIG. 1 and lead 833 is analogous to lead 26 depicted in FIG. 1. The embodiment of FIG. 1 does not have an analogous second lead and electrode 823, 824, but could readily be modified accordingly.

The implantable medical device of FIG. 2 has atrial sensing and pacing channels comprising electrode 834, lead 833, sensing amplifier 831, output circuit 832, and an atrial channel interface 830 which communicates bidirectionally with a port of microprocessor 810. In this embodiment, the device also has ventricular sensing and pacing channels comprising electrodes 824, lead 823, sensing amplifier 821, output circuit 822, and ventricular channel interface 820. For each channel, the same lead and electrode are used for both sensing and pacing. A switch matrix 842 may configure a channel for unipolar sensing or pacing by referencing an electrode of a unipolar or bipolar lead with the implantable medical device housing or can 846. The channel interfaces 820 and 830 include analog-to-digital converters for digitizing sensing signal inputs from the sensing amplifiers and registers which can be written to by the microprocessor in order to output pacing pulses, change the pacing pulse amplitude, and adjust the gain and threshold values for the sensing amplifiers. The implantable medical device can also include one or more sensors 836, such as an accelerometer, a posture sensor, an impedance sensor, a minute ventilation sensor, a pressure sensor, or the like. A telemetry interface 840 is also provided for communicating with a non-implanted device.

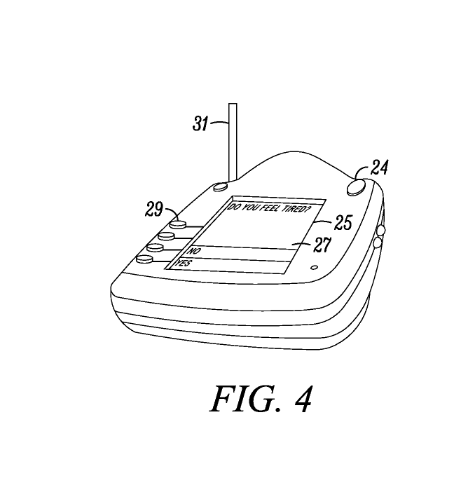

Testing system 20 of FIG. 1 further includes a non-implantable monitor device 24. In some embodiments, monitor device 24 may include the capability of performing functions such as programming of device 22 and recording of data from device 22, and in this case may also be called a programmer/recorder/monitor device 22. Exemplary monitor devices include, but are not limited to, the LATITUDE® patient management system, the Model 2920 Programmer, and the Model 3120 Programmer, each available from Boston Scientific Corporation, Natick, Mass. Aspects of the operation and construction of one embodiment of a monitor device are described in U.S. Published Patent Application 2005/0171411, which is herein incorporated by reference in its entirety. One embodiment of a monitor device 24 is shown in FIG. 4. Monitor device 24 includes an interface 25 that is capable of displaying information and/or messages that can be perceived and understood by a patient. In the embodiment of FIG. 4, the interface 25 includes a screen 27 for displaying information. Monitor device 24 is also capable of receiving input from a patient. In the embodiment, of FIG. 4, monitor device 24 includes buttons 29 and touch sensitive screen 27 for receiving input. Monitor device 24 further includes communication capabilities. Generally, monitor device 24 is capable of communicating with implantable medical device 22 by telemetry, such as by wireless communication path 30. Monitor device 24 includes antenna 31 configured for receiving and transmitting telemetric communications.

Monitor device 24 may also be capable of communicating with a remote computer 32 (also called a remote station 32) through telecommunications, such as over a conventional phone line 34, through cellular phone communications, via the Internet, or any other wired or wireless form of communication. In some embodiments, monitor device 24 is configured to inquire regularly about the patient's general health conditions, such as physical activity and symptoms of disease. Inquiring about the patient's health generally involves displaying one or more health-related questions on an interface such as screen 27 and requesting the patient provide input such as through a touch sensitive screen or buttons 29. The monitor device 24 may also be configured to receive information from the implanted medical device 22 regarding the operation of the device, as well as any information or data stored in the device. The monitor device 24 may be further configured to transmit this information to a remote computer 32, where the information is received and can be further analyzed to determine the patient's medical condition.

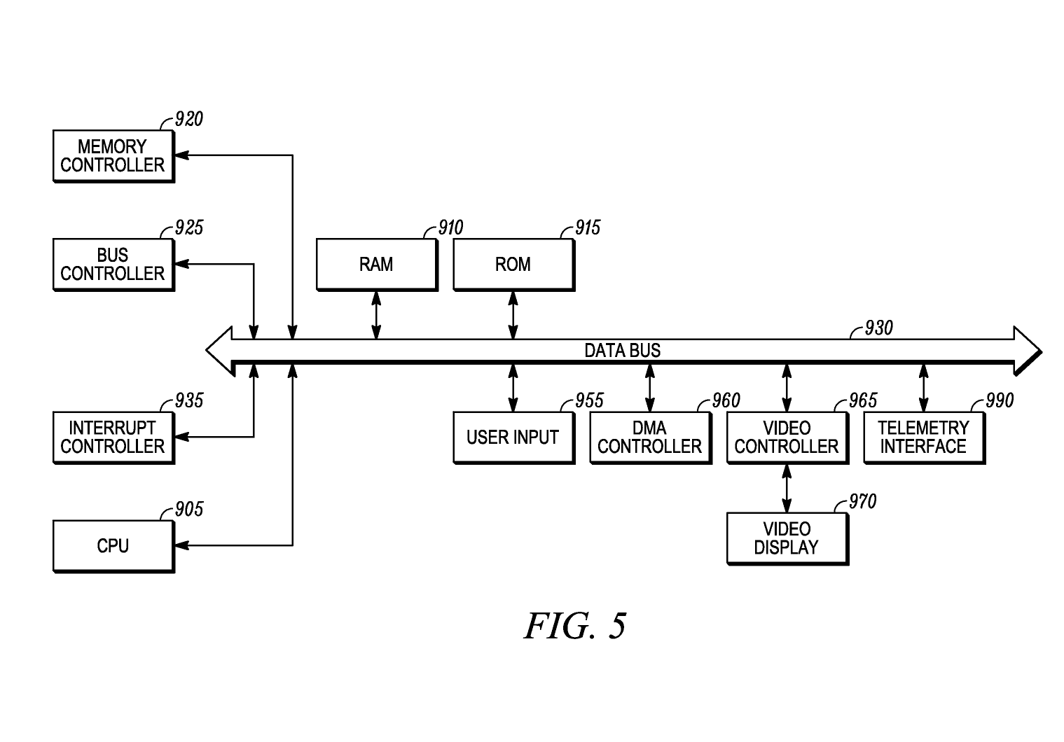

A schematic representation of functional elements of an embodiment of a monitor device 24 is depicted in FIG. 5. Monitor devices can include components common to many computing devices. The monitor device includes a central processing unit (CPU) 905, which may include a conventional microprocessor, random access memory (RAM) 910 for temporary storage of information, and read only memory (ROM) 915 for permanent storage of information. A memory controller 920 is provided for controlling system RAM 910. A bus controller 925 is provided for controlling data bus 930, and an interrupt controller 935 is used for receiving and processing various interrupt signals from the other system components.

User input to the monitor device may be provided by a number of means. For example, user input 955 may be a touch sensitive screen, buttons or keys, voice recognition, or some combination of these. DMA controller 960 is provided for performing direct memory access to system RAM 910. A visual display is generated by a video controller 965, which controls video display 970. Monitor device can also include a telemetry interface 990 which allows the monitor device to interface and exchange data with an implantable medical device.

Testing system 20 of FIG. 1 further includes a plurality of implantable sensors 35 and a plurality of non-implantable sensors 41. In the embodiment of FIG. 1, one of the implantable sensors 35 is a respiration sensor 36. One example of a respiration sensor 36 is an impedance sensor that measures the impedance in the patient's chest region to determine the volumetric rate of respiration. In some embodiments, impedance sensing involves applying low energy pulses (such as about 1 mA of current for 15 microseconds every 50 milliseconds) to an intracardiac lead (such as a ring electrode of a standard bipolar pacing lead) and measuring the resultant voltage between a second intracardiac lead (such as a tip electrode of a standard bipolar pacing lead) and the conductive housing of a CRM device or other implantable medical device located some distance away from the intracardiac leads. The measured transthoracic impedance will increase with inspiration and decrease with exhalation, allowing respiration rate, tidal volume, and minute ventilation to be determined or estimated.

In addition, the embodiment of FIG. 1 includes an implantable posture sensor 38. One example of a posture sensor 38 is an accelerometer, such as a one-axis or three-axis accelerometer, that can determine the orientation of the patient's body with respect to the earth's gravitational field. In addition, the implantable medical device 22 includes electronic circuitry 40 that is capable of monitoring the cardiac electrical signals received from electrode 28 and lead 26. Electronic circuitry 40 is capable of determining a heart rate from the cardiac electrical signals, as well as determining various electrocardiogram intervals, such as R-R interval and QT interval. An R-R interval is generally used to determine the patient's heart rate. A QT interval is generally corrected for heart rate, and is useful for diagnosing the patient's cardiac condition. Some embodiments of testing system 20 further include an implantable blood pressure sensor.

The testing system 20 further includes a plurality of non-implantable sensors 41. In the embodiment of FIG. 1, one of the non-implantable sensors 41 of testing system 20 is a blood pressure tester 42. Blood pressure tester 42 is configured to be used by the patient to measure the patient's blood pressure, such as a cuff that is fitted to the patient's bicep. In the embodiment of FIG. 1, the blood pressure tester 42 is configured to transmit a wireless signal to monitor device 24 that represents the measurement of the patient's blood pressure. In other embodiments, the blood pressure cuff 42 communicates with the monitor device 24 through a wired connection. Generally, non-implantable blood pressure tester 42 includes a cuff that is configured to automatically inflate and deflate at certain time intervals or when manually activated such as with the push of a button. However, in some embodiments, an implantable blood pressure sensor is provided and substitutes for the non-implantable blood pressure sensor.

Testing system 20 is also shown in FIG. 1 as including a non-implantable manometer 44, also called a spirometer 44. Manometer 44 is configured to allow a patient to blow into a mouthpiece, while the manometer monitors the air pressure exerted by the patient. In some embodiments, a timer in the manometer 44 or the monitor device 24 records the amount of time that the patient is blowing into the manometer. In some further embodiments, the manometer provides a signal to the patient, such as an audible signal, that indicates when the patient is blowing with sufficient force (pressure) and when the patient has blown for a sufficient amount of time. Manometer 44 is configured to transmit a signal to monitor device 24 that is representative of the pressure exerted by the patient and time. This transmission may be wireless or wired.

Testing system 20 is further shown in FIG. 1 as including a handgrip dynamometer 45. Handgrip dynamometer 45 has a handle that is configured to be gripped by the patient's hand and to measure the force with which the patient squeezes. In some embodiments, the handgrip dynamometer 45 is configured to provide a signal that indicates the amount of force that the patient is applying. For example, in some circumstances it is desirable that the patient squeeze the dynamometer at a given percentage of a maximum squeezing force, such as 30 percent of maximum. The handgrip dynamometer may be configured to provide an audible or visual signal that assists the patient with maintaining this particular squeezing force. The handgrip dynamometer is configured to transmit a signal to monitor device 24 that represents the force with which the patient is squeezing, or alternatively, is configured to transmit a signal that indicates when the patient is exerting a sufficient amount of force. This transmission may be wired or wireless. In some embodiments a timer in the handgrip dynamometer 45 or monitor device 24 records the length of the squeezing contraction and informs the patient how long they need to maintain the squeezing contraction. Some other embodiments of testing system 20 may also include a body mass scale that is configured to transmit a signal to monitor device 24 that is representative of the patient's body mass. Testing system 20 may include additional non-implantable sensors 41.

In an embodiment, monitor device 24 is programmed or commanded to initiate a CAN test at a regular interval. For example, in one embodiment monitor device 24 is configured to initiate a CAN test at a one month interval, and in another embodiment, to initiate a CAN test at a two month interval, and in yet another embodiment, to initiate a CAN test at a six month interval, and in a further embodiment, to initiate a CAN test at a one year interval. Other intervals are usable. In another embodiment, monitor device 24 initiates a CAN test when commanded to do so, such as by a physician or medical professional.

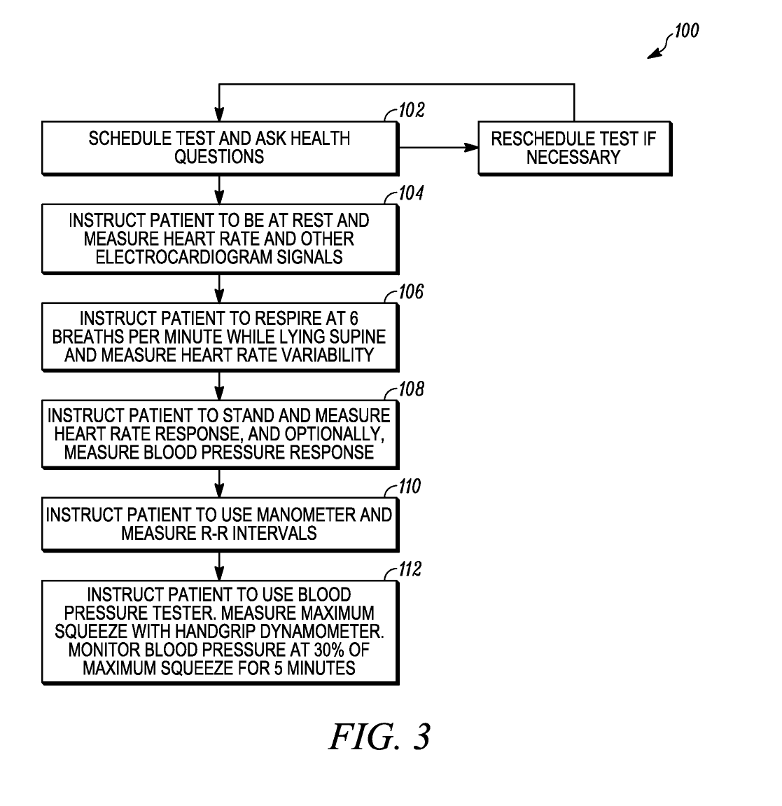

An embodiment of a CAN test protocol is depicted in FIG. 3. Although the steps of one CAN test protocol embodiment are shown in a particular order, the various steps of the testing protocol can be performed in any order and various steps can be omitted or added in other embodiments. CAN test protocol 100 is generally initiated at step 102 by displaying a message on the monitor device 24 that indicates the intent to conduct a test. In one embodiment, a scheduling message is provided so that the patient knows when to expect to perform a CAN test. In some embodiments, the patient is requested to acknowledge the request to perform a CAN test and may be given the option to reschedule the test to a more convenient time. As shown in FIG. 3, if it is necessary to reschedule the test, the test protocol 100 will be initiated at a later time. Once the patient has acknowledged and approved the request to perform the test, the monitor device 24 is generally configured as part of step 102 to ask a series of questions regarding the patient's health and quality of life and to receive input from the patient regarding the answers to the questions. Also, it may be necessary to reschedule the test depending on the answers to the questions. For example, if the patient has not abstained from coffee during a period of time preceding the test, such as eight hours, then the tests for beat-to-beat heart rate variation and heart rate response to standing will be less reliable and should be rescheduled, in one embodiment. These same two tests should also not be performed after an overnight hypoglycemic episode, in one embodiment.

There are many usable embodiments of questions to ask the patient. Examples of appropriate questions are the following:

1. How do you feel today?

2. Do you feel tired?

3. Do you have a difficult time performing physical tasks?

4. Do you occasionally feel out of breath?

5. When did you last drink coffee?

6. Has your blood sugar been difficult to control lately, and if so, when?

7. Have you been lightheaded?

8. Have you had a change in your vision?

9. Have you experienced any burning, tingling, or numbness in your hands or feet?

The monitor device 24 stores the answers to these questions and may transmit them to remote computer 32 at a later time.

After the questions are asked at step 102, the monitor device 24 provides instructions to the patient to perform the various aspects of a CAN screening test. At step 104, the monitor device 24 requests that the patient either sit or lie down and stay at rest for a period of time, and during this time the patient's resting heart rate is recorded. Measuring the patient's resting heart rate involves receiving cardiac electrical signals at electrode 28, which are then passed through lead 26 to electronic circuitry 40 of CRM device 22. Electronic circuitry 40 may be configured to determine a heart rate based on the received cardiac signal and to transmit this information to monitor device 24, or alternatively, the CRM device 22 may transmit the raw cardiac signal to monitor device 24 where the heart rate is determined. In one embodiment, the electronic circuitry 40 determines the heart rate based on an interval in the electrocardiogram, such as an R-R interval. In another embodiment, the electronic circuitry determines the QT interval from the electrocardiogram and corrects the determined QT interval for the heart rate (denoted QTc).

At step 106, the monitor device 24 requests that the patient lie supine (on the back) while breathing at the rate of six breaths per minute. In some embodiments, an implantable respiration sensor 36 is consulted to verify that the patient is breathing at an appropriate rate. The respiration sensor may be a trans-thoracic respiration sensor 36 or an accelerometer 38, or a variety of other sensors that provide information about a patient's respiration. In one embodiment, the monitor device 24 assists the patient in breathing at a rate of six breaths per minute by providing an audible signal once every 10 seconds, such that the patient can use the audible signal to time each breath. The patient's heart rate and heart rate variability is then measured. This can be accomplished in electronic circuitry 40, which may be configured to measure intervals on an electrocardiogram. For example, the circuitry 40 may measure an R-R interval after the patient has slowed breathing, and compare the R-R interval to the R-R interval before the patient's breathing was slowed. The circuitry 40 also determines the beat-to-beat variability in one embodiment. In addition to measuring the heart rate variability over the course of the test, the circuitry 40 may be configured to measure the patient's R-R interval during exhalation and also during inspiration. The heart rate variability and ratio of exhalation to inspiration R-R values is transmitted to monitor device 24. Alternatively, raw cardiac signal data may be transmitted to the monitor device 24, where the appropriate cardiac intervals and ratios are determined.

At step 108, the monitor device 24 requests that the patient sit down or lie down for a period of time, such as 30 seconds, and then stand up and remain standing for a period of time, such as 30 seconds or more. Other time intervals are usable, however, and in some embodiments, it is necessary for the patient to remain standing longer than 30 seconds. In some embodiments, the implantable posture sensor 38 is used to determine when the patient has transitioned from a sitting position to a standing position. At the point that the patient is standing, electronic circuitry monitors the patient's cardiac activity. A typical data collection involves measuring the R-R interval at the 15^{th} beat after standing and at the 30^{th} beat after standing. This information is then transmitted to monitor device 24, or alternatively, the raw cardiac data is transmitted to monitor device 24 and the data is analyzed in the monitor device 24.

In one embodiment, step 108 also includes instructing the patient to utilize non-implantable blood pressure tester 42, where blood pressure tester 42 is present, prior to beginning the procedure. Instructing the patient to use the blood pressure tester 42 generally includes instructing the patient to place a blood pressure cuff on the patient's bicep or other suitable location. In some embodiments, however, a non-implantable blood pressure tester 42 is not used, but instead an implantable blood pressure sensor is available. In either case, step 108 further includes the step of instructing the patient to remain standing for a period of time, such as two minutes. Then blood pressure measurements are taken either by blood pressure tester 42 or by implantable blood pressure tester, at a first time while the patient is sitting or lying down and at a second time two minutes after standing. If a non-implantable blood pressure tester 42 is used, it is configured to automatically inflate and deflate at the appropriate times in order to take a blood pressure measurement. The blood pressure measurements are transmitted to monitor device 24. Alternatively, the change in the patient's blood pressure from the first time to the second time is determined and the calculated difference is transmitted to monitor device 24.

At step 110, the monitor device 24 instructs the patient to use non-implantable manometer 44. The patient is generally instructed to forcibly exhale into a mouthpiece of the manometer 44 and to maintain a pressure of at least 40 mm Hg for at least 15 seconds. As described above, the manometer 44 or the monitor device 24 may provide indications to assist the patient with meeting these requirements, such as an audible signal that indicates that sufficient pressure is achieved and a separate audible signal that indicates that sufficient time has passed. During the 15 seconds where the patient is exhaling at the appropriate pressure, electronic circuitry 40 is configured to measure the R-R interval of each heart beat. The R-R intervals are transmitted to monitor device 24, or alternatively, raw cardiac signal data is transmitted to monitor device 24 where the R-R intervals are determined.

At step 112, the monitor device 24 instructs the patient to use the handgrip dynamometer 45, if available, along with the blood pressure tester 42, if available. The patient is instructed to apply a cuff of the blood pressure tester to one arm if a non-implantable blood pressure tester 42 is used. The patient is then instructed to squeeze the handgrip dynamometer 45 as hard as possible with the arm opposite to the one that has the blood pressure tester 42 on it. This procedure is used to define the patient's maximum handgrip force. Then a value that represents 30 percent of the patient's maximum handgrip force is calculated. The patient is then instructed to squeeze the handgrip dynamometer 45 with the same hand as used to determine the maximum at the 30 percent of maximum value for 5 minutes. In some embodiments, monitor device 24 includes a timer to assist the patient in determining when the appropriate time interval has elapsed and may provide an audible signal to indicate that the patient can relax. While the patient is squeezing the handgrip dynamometer 45, the blood pressure tester 42 monitors the patient's blood pressure in the contralateral arm. This blood pressure information is transmitted to control device 24.

In each of steps 102 to 112, data is generated concerning the patient's performance or response to each test. This data may be transmitted in a raw or unprocessed form to monitor device 24, or may be processed in an implantable or non-implantable device and then transmitted to monitor device 24 in a summary condition.

In one embodiment, the data generated from testing protocol 100 is analyzed in monitor device 24 to determine whether the patient has symptoms of CAN, and if so, the nature and extent of the symptoms. In another embodiment, the data generated from testing protocol 100 is transmitted to remote computer 32, where it is analyzed to determine whether the patient has symptoms of CAN and the nature and extent of the symptoms. In yet another embodiment, the data generated from testing protocol 100 is transmitted to remote computer 32, where it is presented to a trained medical person who can evaluate it to determine whether the patient has symptoms of CAN and the extent of the symptoms and progression of CAN. Further, the data may be analyzed by a combination of these ways or in all of these ways.

In embodiments where the data from testing protocol 100 is analyzed in monitor device 24 or remote computer 32, parameters may be programmed into or stored within the respective device to form a basis for making an evaluation of CAN. In one aspect of the data analysis, a parameter is provided that is associated with the data from step 104, such that a resting heart rate of greater than 100 beats per minute is an indication of the presence of CAN. Other parameters may be provided that are associated with other aspects of the patient's electrocardiogram, such as a QTc of more than 440 ms.

Another parameter is a heart-rate variability having depressed very-low frequency peak, a depressed high frequency peak, or a lowered low-frequency to high-frequency ratio, where the presence of one or more of these conditions is an indication of the presence of CAN, where these parameters are typically determined by a frequency transform of electrocardiogram data over an extended period, such as a 24 hour period. A description of methods of analyzing heart rate variability for assessing autonomic balance is described in commonly-owned U.S. Pat. Nos. 7,069,070, 7,215,992 and U.S. Published Patent Application 2004-0158295, which patent documents were previously incorporated herein by reference.

In another aspect of the data analysis, a parameter is provided that is associated with the data from step 106, such that a difference in heart rate of less than 10 beats per minute while the patient breaths at the rate of six breaths per minute, and an exhalation to inspiration ratio of greater than 1.17, is an indication of the presence of CAN. In a further aspect of the data analysis, a parameter is provided that is associated with the data from step 108, such that a ratio of the R-R interval at the 30^{th} beat to the R-R ratio at the 15^{th} beat after standing is less than 1.03, and a fall in blood pressure of more than 30 mm Hg, or in some cases more than 10 mm Hg, is an indication of the presence of CAN. In yet another aspect of the data analysis, a parameter is provided that is associated with the data from step 110, such that a ratio of the longest R-R interval to the shortest R-R interval during the Valsalva maneuver of less than 1.2 is an indication of the presence of CAN. In a further aspect of the data analysis, a parameter is provided that is associated with the data from step 112, such that a rise of less than 16 mm Hg in the contralateral arm is an indication of the presence of CAN.

In some embodiments, the determination of whether the patient has CAN is made where any one of the parameters provide an indication of the presence of CAN. In other embodiments, the number of parameters that provide an indication of the presence of CAN and/or the degree to which the patient deviates from the parameters can be used to provide an indication of the extent or progression of the patient's CAN. In some embodiments, where any parameter indicates the presence of CAN, a trained medical person is alerted and analyzes the data further.

In some embodiments, certain testing steps can be done without the patient knowing and without the patient's involvement. For example, a heart rate measurement may be taken any time the posture sensor indicates an appropriate, sudden change in posture, such as would be representative of the patient standing up from a sitting position.

In some embodiments, less than all of the steps of testing protocol 100 shown in FIG. 3 are performed. There are certain steps that may be more useful in providing an indication of CAN and therefore may be retained in an embodiment that includes less than all of the steps shown in FIG. 3. For example, one usable embodiment of a testing protocol could include only steps 106 and 108, where the patient's heart rate variability is measured while breathing at a rate of six breaths per minute and where the patient's heart rate and blood pressure response to standing are measured. However, other combinations of steps are usable.

In one embodiment, the monitor device 24 can be programmed to customize which tests are administered to the patient. For example, a physician may program monitor device 24 to administer certain tests and to not administer other tests.

The present invention should not be considered limited to the particular examples described above, but rather should be understood to cover all aspects of the invention as fairly set out in the attached claims. Various modifications, equivalent processes, as well as numerous structures to which the present invention may be applicable will be readily apparent to those of skill in the art to which the present invention is directed upon review of the present specification. The claims are intended to cover such modifications and devices.

The above specification provides a complete description of the structure and use of the invention. Since many of the embodiments of the invention can be made without parting from the spirit and scope of the invention, the invention resides in the claims.

What is claimed is:

1. A system for performing cardiovascular autonomic neuropathy (CAN) testing in a diabetic patient, the system comprising:
(i) an implantable medical device (IMD) that includes a plurality of implantable physiological sensors, the IMD being configured to:
(a) transmit a wireless signal corresponding to a sensed physiological activity; and
(b) receive wireless signals;
(ii) one or more non-implantable physiological sensors, where the non-implantable physiological sensors are each configured to transmit a signal corresponding to a sensed physiological parameter; and
(iii) a monitor device having a patient interface and configured to:
(a) interface with a patient, including directing the patient to
(A) answer health related questions; and
(B) use one or more of the non-implantable physiological sensors; and
(b) receive signals, including signals from the IMD and the non-implantable physiological sensors; and
(iv) wherein the system is configured to provide an indication of the presence or progression of CAN.

2. The system of claim 1, where the implantable physiological sensors comprise a minute ventilation sensor.

3. The system of claim 2, where the minute ventilation sensor comprises a trans-thoracic impedance sensor.

4. The system of claim 1, where the implantable physiological sensors comprise a body posture sensor.

5. The system of claim 4, where the body posture sensor comprises an accelerometer.

6. The system of claim 1, where the implantable physiological sensors comprise an instantaneous heart rate sensor.

7. The system of claim 1, where the implantable physiological sensors comprise an implantable blood pressure sensor.

8. The system of claim 1, where the non-implantable physiological sensors comprise a blood pressure measurement device.

9. The system of claim 1, where the non-implantable physiological sensors comprise a hand grip dynamometer.

10. The system of claim 1, where the non-implantable physiological sensors comprise a spirometer.

11. The system of claim 1, where the monitor device is configured to transmit a signal to a remote station.

12. The system of claim 11, where the remote station is configured to analyze the received signal to determine the presence or progession of CAN in a patient.

13. A method of testing for cardiovascular autonomic neuropathy (CAN) in a diabetic patient, the method comprising:
(i) providing an implanted medical device (IMD) that includes a plurality of implantable physiological sensors, the IMD being configured to
(a) transmit a wireless signal corresponding to a sensed physiological activity; and
(b) receive wireless signals;
(ii) providing a plurality of non-implantable physiological sensors, where the non-implantable physiological sensors are each configured to transmit a signal corresponding to a sensed physiological parameter;
(iii) providing a monitor device having a patient interface;
(iv) directing a patient through the patient interface to:
(a) answer health related questions;
(b) interface with one or more of the non-implantable physiological sensors; and
(c) perform a diagnostic procedure;
(v) receiving signals at the monitor device, including signals from the IMD and the non-implantable physiological sensors; and
(vi) using an implantable physiological sensor to verify that the patient properly performed the diagnostic procedure.

14. The method of claim 13, further including the step of processing the signals at the monitor device to determine the presence or progression of CAN.

15. The method of claim 13, further including the step of transmitting data corresponding to the signals received at the monitor to a remote station for processing and to determine the presence or progression of CAN.

16. The method of claim 13, where the monitor device is configured to transmit a signal to a remote station.

17. The method of claim 13, where directing the patient to perform a diagnostic procedure includes directing the patient to lie supine and then stand.

18. The method of claim 17, where verifying that the patient performed the diagnostic procedure includes using the signal from an implantable accelerometer.

19. The method of claim 13, where directing the patient to perform a diagnostic procedure includes instructing the patient to breathe at a rate of six breaths per minute.

20. The method of claim 19, where verifying that the patient performed the diagnostic procedure includes using the signal from an implantable respiration sensor.

21. A system for performing testing for cardiovascular autonomic neuropathy (CAN) in a diabetic patient, the system comprising:
(i) an implanted medical device (IMD) system that includes a plurality of implantable physiological sensors, the IMD system being configured to
(a) transmit a wireless signal corresponding to a sensed physiological activity; and
(b) receive wireless signals;
(ii) a plurality of non-implantable physiological sensors, where the non-implantable physiological sensors are each configured to transmit a signal corresponding to a sensed physiological parameter;
(iii) a monitor device configured to receive signals including signals from the IMD system and the non-implantable physiological sensors, the monitor device comprising a patient interface and configured to direct a patient through the patient interface to:
(a) answer health related questions;
(b) interface with one or more of the non-implantable physiological sensors; and
(c) perform a diagnostic procedure;
(iv) wherein the IMD system further comprises an implantable physiological sensor to verify that the patient properly performed the diagnostic procedure.

22. The system of claim 21 wherein the implantable physiological sensor includes an accelerometer.

23. The system of claim 21 wherein the implantable physiological sensor includes a body posture sensor.

24. The system of claim 21 wherein the implantable physiological sensor includes an implantable respiration sensor.
